(19) United States
(12) Patent Application Publication
(10) Pub. No.: US 2003/0083342 A1
(43) Pub. Date: May 1, 2003

(54) Combination of organic compounds

(76) Inventor: Ronald Edward Steele, Long Valley, NJ (US)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/149,107

(22) PCT Filed: Apr. 10, 2001
(86) PCT No.: PCT/EP01/04116

(51) Int. Cl. A61K 31/4745

(52) U.S. Cl. 514/303

(57) ABSTRACT

The invention relates to a pharmaceutical composition, of (i) an aldosterone synthase inhibitor or a pharmaceutically acceptable salt thereofeither alone or in combination with (ii) an AT₁-receptor antagonist combined with a diuretic, or in each case, a pharmaceutically acceptable salt thereof and (iii) a pharmaceutically acceptable carrier.

The invention relates to a pharmaceutical composition comprising

(i) an aldosterone synthase inhibitor or a pharmaceutically acceptable salt thereof alone or in combination with,

(ii) an AT₁-receptor antagonist or an AT₁ receptor antagonist combined with a diuretic or, in each case, a pharmaceutically acceptable salt thereof and

(iii) a pharmaceutically acceptable carrier. The invention furthermore relates to a method for the prevention of, delay of progression of, treatment of a disease or condition selected from the group consisting of

(a) hypertension, congestive heart failure, renal failure, especially chronic renal failure, restenosis after percutaneous transluminal angioplasty, and restenosis after coronary artery bypass surgery;

(b) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and

(c) endothelial dysfunction with or without hypertension, comprising administering the pharmaceutical composition of the present invention.

In a preferred embodiment the present invention relates to a method of prevention of, delay of progression of or treatment of endothelial dysfunction with or without hypertension comprising administering to a warm-blooded animal, including man, in need thereof an effective amount of an aldosterone synthase inhibitor or a pharmaceutically acceptable salt thereof.

AT₁-receptor antagonists (also called angiotensin II receptor antagonists) are understood to be those active ingredients which bind to the AT₁-receptor subtype of angiotensin 11 receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds which are selected from the group consisting of valsartan, losartan, candesartan, eprosartan, irbesartan, saprisartan, tasosartan, telmisartan, the compound with the designation E-1477 of the following formula

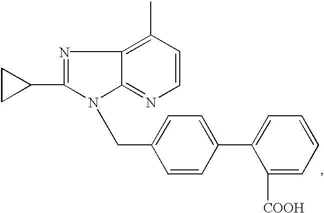

the compound with the designation SC-52458 of the following formula

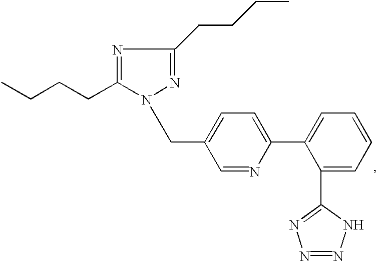

and the compound with the designation the compound ZD-8731 of the following formula

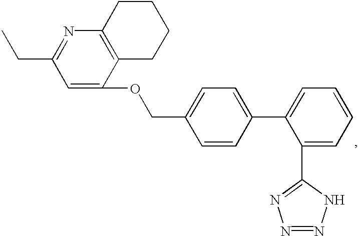

or, in each case, a pharmaceutically acceptable salt thereof.

Preferred AT₁-receptor antagonist are those agents which have been marketed, most preferred is valsartan or a pharmaceutically acceptable salt thereof.

A diuretic is, for example, a thiazide derivative selected from the group consisting of chlorothiazide, hydrochlorothiazide, methylclothiazide, and chlorothalidon. The most preferred is hydrochlorothiazide.

Aldosterone synthase inhibitor is an enzyme which converts corticosterone to aldosterone to by hydroxylating cortocosterone to form 18-OH-corticosterone and 18-OH-corticosterone to aldosterone. The class of aldosterone synthase inhibitors know to be applied for the treatment of hypertension and primary aldosteronism comprises both steroidal and non-steroidal aldosterone synthase inhibitors, the later being most preferred.

Preference is given to commercially available aldosterone synthase inhibitors or those aldosterone synthase inhibitors that have been approved by the health authorities. The class of aldosterone synthase inhibitors comprises compounds having differing structural features. For example, mention may be made of the compounds which are selected from the group consisting of the non-steroidal aromatase inhibitors anastrozole, fadrozole (including the (+)-enantiomer thereof), as well as the steroidal aromatase inhibitor exemestane, or, in each case where applicable, a pharmaceutically acceptable salt thereof.

The most preferred non-steroidal aldosterone synthase inhibitor is the (+)-enantiomer of the hydrochloride of fadrozole (U.S. Pat. Nos. 4617307 and 4889861) of formula

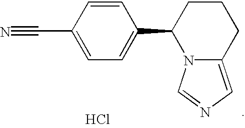

Surprisingly, the pharmaceutical compositions according the present invention exhibit a beneficial, especially a synergistic (=more than additive), therapeutic effect, furthermore benefits resulting from combined treatment such as a surprising prolongation of efficacy, a broader variety of therapeutic treatment and surprising beneficial effects on diseases and conditions associated with AT₁-receptors or aldosterone synthase inhibitors, respectively.

The compositions according to the present invention can be used for the prevention of, the delay of progression of and treatment of a disease or condition selected from the group consisting of

(a) hypertension, congestive heart failure, renal failure, especially chronic renal failure, restenosis after percutaneous transluminal angioplasty, and restenosis after coronary artery bypass surgery;

(b) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and

(c) endothelial dysfunction with or without hypertension.

The person skilled in the pertinent art is fully enabled to select a relevant animal test model to prove the hereinbefore and hereinafter indicated therapeutic indications and beneficial effects.

These beneficial effects can, for example, be demonstrated in the test model as disclosed by G. Jeremic et al. in J. Cardovasc. Pharmacol. 27:347-354, 1996.

### Study design

In the study to be performed, permanent coronary artery occlusion (CAO) in rats is used as a model of acute myocardial infarction. The experiments are carried out with 5 treatment groups characterized by following features:

sham-operated animals

CAO+vehicle

CAO+valsartan

CAO+aldosterone synthase inhibitor

CAO+AT₁-receptor antagonist+aldosterone synthase inhibitor.

Following doses and routes of administration can be applied:

For example for the AT,-receptor antagonist valsartan

a) −3d to +2d: s.c. injections 2.5 mg/kg BW/12 h

b) +3d to +28d: s.c. Alza osmotic minipumps 5 mg/kg/d

For the (+)-enantioner of the hydrochloride of fadrozole Alza osmotic minipumps 0.4 mg/kg/d.

During the study following variables are measured:

infarct size

LV chamber volume

interstitial and perivascular collagen density in spared LV myocardium

COL-I and COL-III protein content in spared LV myocardium by Western blot

cardiomyocytes cross-sectional area and length in sections of LV myocardium

plasma concentrations of Ang II and aldosterone

urine concentration of sodium, potassium and aldosterone

blood pressure in conscious animals

LV and carotid blood pressure in anesthetized animals.

### Methodology

Infarct size: Six μm-thick transverse histological sections of the left ventricle are stained with nitroblue tetrazolium and acquired by a B/W XC-77CE CCD video camera (Sony). The resulting image is processed on a KS 300 image analysis system (Carl Zeiss Vision) using a software specifically developed (Porzio et al., 1995). A single operator blinded to treatment interactively defines the boundaries of the interventricular septum, and the infarcted area on each section is semiautomatically identified as the area of unstained ventricular tissue. The software automatically calculates for each component of the ventricular section defined as the chamber, septum, infarcted area, infarcted LV wall and viable LV wall, a set of geometric parameters (Porzio et al., 1995).

Histology: Hearts are fixed in situ, by retrograde perfusion with buffered 4% formaldehyde after arrest in diastole by i.v. injection of 0.5 M KCI. After fixation, the left ventricle (LV) and the free wall of the right ventricle are separately weighed; LV longer diameter is measured with a caliper. LV histological sections are stained with hematoxylin & eosin for qualitative examination and to quantify cardiomyocytes cross-sectional area with a semi-automated image analysis routine. Interstitial collagen deposition in LV is evaluated on Sirius red stained sections with a semi-automated image analysis routine (Masson et al., 1998).

Collagen content in LV spared myocardium: LV tissue in the spared myocardium is homogenized, subjected to PAGE-SDS electrophoresis and electroblotted onto nitrocellulose membrane. The blots are exposed to primary antibodies, i.e. rabbit anti-rat collagen type I or type III antiserum (Chemicon). The primary antibodies are recognized by secondary antibodies conjugated to alkaline phosphatase (for collagen type 1) or peroxidase (collagen type II).

Left ventricular chamber volume: LV chamber volume is determined in hearts arrested in diastole (KCI) and fixed in formalin under a hydrostatic pressure equivalent to the measured LV end-diastolic pressure. A metric rod is inserted into the LV to measure LV inner length. The transverse diameters of the LV chamber are measured in two 1-mm thick transverse sections near to the base and the apex of the ventricle (Jeremic et al, 1996). The chamber volume is computed from an equation integrating transverse diameters and ineer length.

Systemic and Left ventricular hemodynamics: A microtip pressure transducer (Millar SPC-320) connected to a recorder (Windograf, Gould Electronics) is inserted into the right carotid artery to record systolic and diastolic blood pressures. The pressure transducer is advanced into the LV to measure LV systolic (LVSP) and end-diastolic (LVEDP) pressures, the first derivative of LV pressure over time (+dP/dt) and heart rate.

Non-invasive blood pressure: Systolic blood pressure and heart rate are measured by the tail-cuff method (Letica LE 5002) in conscious rats.

Urine electrolytes, hormones: Rats are individually housed in metabolic cages and 24-h urine collected on 1 ml HCI 6N. Water intake is measured. Urine catecholamines are extracted on Bondelut C₁₈ columns (Varian), separated by HPLC (Apex-II C18, 3 μm, 50×4.5 mm analytical column, Jones Chromatography) and quantified with an electrochemical detector (Coulochem II, ESA) (Goldstein et al., 1981). Plasma and urine aldosterone, and plasma angiotensin II is determined with specific radioimmunoassays (Aldoctk-2, DiaSorin and Angiotensin II, Nichols Diagnostics). Urine sodium and potassium are measured by flamme photometry.

### Sample size

10 animals analyzable in each treatment groups are sufficient to detect biologically significant differences. Only rats with an infarct size of at least 10% of the LV section area are included in the final analysis.

Accordingly, the composition of the present invention can be used for the prevention of, delay of progression of, and treatment of survival post myocardial infarction (Ml).

Endothelial dysfunction is being acknowledged as a critical factor in vascular diseases. The endothelium plays a bimodal role as the source of various hormones or by-products with opposing effects: vasodilation and vasoconstriction, inhibition or promotion of growth, fibrinolysis or thrombogenesis, production of anti-oxidants or oxidising agents. Genetically predisposed hypertensive animals with endothelial dysfunction constitute a valid model for assessing the efficacy of a cardiovascular therapy.

Endothelial dysfunction is characterized by, for example, increased oxidative stress, causing decreased nitric oxide, increased factors involved in coagulation or fibrinolysis such as plasminogen activating inhibitor-1 (PAI-1), tissue factor (TF), tissue plasminogen activator (tPA), increased adhesion molecules such as ICAM and VCAM, increased growth factors such as bFGF, TGFβ, PDGF, VEGF, all factors causing cell growth, inflammation and fibrosis.

The treatment e.g. of endothelial dysfunction can be demonstrated in the following pharmacological test:

### Material and methods

Male 20-24 week-old SHR, purchased from RCC Ldt (Fullingsdorf, Switzerland), are maintained in a temperature- and light-controlled room with free access to rat chow (Nafag 9331, Gossau, Switzerland) and tap water. The experiment is performed in accordance with the NIH guidelines and approved by the Canton Veterinary office (Bew 161, Kantonales Veterinaramt, Liestal, Switzerland). All rats are treated with the NO synthase inhibitor L-NAME (Sigma Chemicals) administered in drinking water (50 mg/l) for 12 weeks. The average daily dose of L-NAME calculated from the water consumed was 2.5 mg/kg/d (range 2.1-2.7).

The rats are divided into 5 groups: group 1, control (n=40); Group 2, valsartan (5 mg/kg/d; n=40); Group 3, the (+)-enantiomer of the hydrochloride of fadrozole (n=30); Group 4, a combination of the (+)-enantiomer of the hydrochloride of fadrozole and valsartan (5 mg/kg/d); n=30) and Group 5, valsartan (50 mg/kg/d; n=30). The drugs are administered in drinking fluid. The pressor effect of Ang II at 1 mg/kg obtained in controls normotensive rats is reducted by 49% and 73% after treatment with valsartan 5 and 50 mg/kg/d, respectively (Gervais et al. 1999). The response to Ang I injected in Wistar Kyoto rats pretreated with the (+)-enantiomer of the hydrochloride of fadrozole or valsartan 5 mg/kg/d is similar.

Body weight is measured every week. Systolic blood pressure and heart rate are recorded by tail cuff plethysmography 3 and 2 weeks before starting the study and at 2 weeks after drug administration. Urine is collected over a 24 hour period from rats kept in individual (metabolic) cages the week before starting treatment and at weeks 4 and 12 for volume measurement and protein, creatinine, sodium and potassium determination using standard laboratory methods. At the same time points, blood samples are withdrawn from the retro-orbital plexus (maximum 1 ml) for creatinine, Na+and K+assays.

Ten rats from each group are sacrificed at 4 weeks for collection of kidney and heart for morphological analysis. The remaining rats are sacrificed at 12 weeks. Cardiac and kidney weight is recorded. Terminal blood sampling is performed in 5% EDTA at 4 (morphometry study) and 12 (end of the study) weeks for aldosterone, determination by radioimmunoassay using a DPC coat-a-count aldosterone-RIA kit (Buhlmann, Switzerland).

### Statistical analysis:

All data are expressed as mean +SEM. Statistical analysis is performed using a one-way ANOVA, followed by a Duncan's multiple range test and a Newman-Keuls test, for comparison between the different groups. Results with a probability value of less than 0.05 are deemed statistically significant.

An improvement of regression of atherosclerosis without effecting the serum lipid levels can, for exmple, be demonstrated by using the animal model as disclosed by H. Kano et al. in Biochemical and Biophysical Research Communications 259, 414-419 (1999).

That the compounds or combinations according to the present invention can be used for the regression of a cholesterol diet-induced atherosclerosis, can be demonstrated using the test model described, e.g., by C. Jiang et al. in Br. J. Pharmacol. (1991), 104, 1033-1037.

That the compounds or compositions according to the present invention can be used for the treatment of renal failure, especially chronic renal failure, can be demonstrated using the test model described, e.g., by D. Cohen et al. in Journal of Cardiovascular Pharmacology, 32: 87-95 (1998).

Further benefits when applying the composition of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

All the more surprising is the experimental finding that the combined administration of combination according to the present invention results in a beneficial, especially a synergistic, therapeutic effect, but also in benefits resulting from the combined treatment and further surprising beneficial effects compared to a monotherapy applying only one of the pharmaceutically active compounds used in the combinations disclosed herein. In particular, all the more surprising is the experimental finding that the combination of the present invention results in a beneficial, especially a synergistic, therapeutic effect but also in benefits resulting from combined treatment such as a surprising prolongation of efficacy, a broader variety of therapeutic treatment and surprising beneficial effects on diseases and conditions as specified hereinbefore or hereinafter.

Further benefits when applying the composition of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

The results of the studies clearly show that the composition according to the present invention can be used for the prevention of, delay of progression of, treatment of a disease or condition selected from the group consisting of

(a) hypertension, congestive heart failure, renal failure, especially chronic renal failure, restenosis after percutaneous transluminal angioplasty, and restenosis after coronary artery bypass surgery;

(b) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and

(c) endothelial dysfunction with or without hypertension.

The compositions of the present invention can also be used for the prevention and delay of progression and preferably the treatment of other diseases.

A preferred composition comprises the combination of the (+)-enantiomer of the hydrochloride of fadrozole and valsartan or valsartan combined with hydrochlorothiazide. Preferably, the jointly therapeutically effective amounts of an AT₁-receptor antagonist or of an AT₁-receptor antagonist combined with a diuretic, in each case, in free or pharmaceutically acceptable salt form and an aldosterone synthase inhibitor in free or pharmaceutically acceptable salt form can be administered simultaneously or sequentially in any order, separately or in a fixed combination.

Furthermore, the invention relates to a method of the prevention of, delay of progression of, treatment of a disease or condition selected from the group consisting of

(a) hypertension, congestive heart failure, renal failure, especially chronic renal failure, restenosis after percutaneous transluminal angioplasty, and restenosis after coronary artery bypass surgery;

(b) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and

(c) endothelial dysfunction with or without hypertension; comprising administering to a warm-blooded animal, including man, a therapeutically effective amount of an aldosterone synthase inhibitor in free or pharmaceutically acceptable salt form either alone or in combination with an AT₁-receptor antagonist or in combination with an AT₁-receptor antagonist combined with a diuretic, in each case, in free or pharmaceutically acceptable salt form.

Furthermore, the invention relates to the use of a

(a) pharmaceutical composition comprising

(i) an AT,-receptor antagonist or an AT₁-receptor antagonist combined with a diuretic, or, in each case, a pharmaceutically acceptable salt thereof,

(ii) an aldosterone synthase inhibitor or a pharmaceutically acceptable salt thereof and

(iii) a pharmaceutically acceptable carrier; or

(b) an aldosterone synthase inhibitor or a pharmaceutically acceptable salt thereof, for the manufacture of a medicament for the prevention of, delay of progression of, treatment of a disease or condition selected from the group consisting of

(α) hypertension, congestive heart failure, renal failure, especially chronic renal failure, restenosis after percutaneous transluminal angioplasty, and restenosis after coronary artery bypass surgery;

(β) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and

(χ) endothelial dysfunction with or without hypertension.

The present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The invention furthermore relates to a commercial package comprising the combination according to the present invention together with instructions for simultaneous, separate or sequential use.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner which is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commerically available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

Valsartan, as a representative of the class of AT₁-receptor antagonists, will be supplied in the form of suitable dosage unit form, for example, a capsule or tablet, and comprising a therapeutically effective amount, e.g. from about 20 to about 320 mg, of valsartan which may be applied to patients. The application of the active ingredient may occur up to three times a day, starting e.g. with a daily dose of 20 mg or 40 mg of valsartan, increasing via 80 mg daily and further to 160 mg daily up to 320 mg daily. Preferably, valsartan is applied twice a day with a dose of 80 mg or 160 mg, respectively, each. Corresponding doses may be taken, for example, in the moming, at mid-day or in the evening.

The following examples illustrate the above-described invention; however, it is not intended to restrict the scope of this invention in any manner.

FORMULATION EXAMPLE 1
Film-Coated Tablets:

| | Composition Per | |
|---|---|---|
| Components | Unit (mg) | Standards |
| Granulation | | |
| Valsartan [= active ingredient] | 80.00 | |
| Microcrystalline cellulose/ | 54.00 | NF, Ph. Eur |
| Avicel PH 102 | | |
| Crospovidone | 20.00 | NF, Ph. Eur |
| Colloidal anhydrous silica/ | 0.75 | Ph. Eur/ |
| colloidal silicon dioxide/Aerosil 200 | | NF |
| Magnesium stearate | 2.5 | NF, Ph. Eur |
| Blending | | |
| Colloidal anhydrous silica/ | 0.75 | Ph. Eur/ |
| colloidal silicon dioxide/Aerosil 200 | | NF |
| Magnesium stearate | 2.00 | NF, Ph. Eur |
| Coating | | |
| Purified water*⁾ | — | |
| DIOLACK pale red 00F34899 | 7.00 | |
| Total tablet mass | 167.00 | |

*⁾Removed during processing.

The film-coated tablet is manufactured e.g. as follows:

A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screnning mill. The resulting mixture is again pre-mixed in a diffusion mixer, compacted in a roller compacter and then sieve through a screening mill. To the resulting mixture, the rest of the colloidal anhydrous silicalcolloidal silicon dioxide/Aerosile 200 are added and the final blend is made in a diffusion mixer. The whole mixture is compressed in a rotary tabletting machine and the tabletts are coated with a film by using Diolack pale red in a perforated pan.

FORMULATION EXAMPLE 2
Film-coated tablets:

| | Composition Per | |
|---|---|---|
| Components | Unit (mg) | Standards |
| Granulation | | |
| Valsartan [= active ingredient] | 160.00 | |
| Microcrystalline cellulose/ | 108.00 | NF, Ph. Eur |
| Avicel PH 102 | | |
| Crospovidone | 40.00 | NF, Ph. Eur |
| Colloidal anhydrous silica/ | 1.50 | Ph. Eur/ |
| colloidal silicon dioxide/Aerosil 200 | | NF |
| Magnesium stearate | 5.00 | NF, Ph. Eur |
| Blending | | |
| Colloidal anhydrous silica/ | 1.50 | Ph. Eur/ |
| colloidal silicon dioxide/Aerosil 200 | | NF |
| Magnesium stearate | 4.00 | NF, Ph. Eur |
| Coating | | |
| Opadry Light Brown 00F33172 | 10.00 | |
| Total tablet mass | 330.00 | |

The film-coated tablet is manufactured e.g. as described in Formulation Example 1.

FORMULATION EXAMPLE 3
Film-Coated Tablets:

| | Composition Per | |
|---|---|---|
| Components | Unit (mg) | Standards |
| Core Internal phase | | |
| Valsartan | 40.00 | |
| [= active ingredient] | | |
| Silica, colloidal anhydrous | 1.00 | Ph. Eur, USP/NF |
| (Colloidal silicon dioxide) | | |
| [= Glidant] | | |
| Magnesium stearate | 2.00 | USP/NF |
| [= Lubricant] | | |
| Crospovidone | 20.00 | Ph. Eur |
| [Disintegrant] | | |
| Microcrystalline cellulose | 124.00 | USP/NF |
| [= Binding agent] | | |
| External phase | | |
| Silica, colloidal anhydrous, | 1.00 | Ph. Eur, USP/NF |
| (Colloidal silicon dioxide) | | |
| [= Glidant] | | |
| Magnesium stearate | 2.00 | USP/NF |
| [Lubricant] | | |
| Film coating | | |
| Opadry ® brown OOF 16711*⁾ | 9.40 | |
| Purified Water**⁾ | — | |
| Total tablet mass | 199.44 | |

*⁾The composition of the Opadry ® brown OOF16711 coloring agent is tabulated below.
**⁾Removed during processing

Opadry ® Composition:

| | Approximate % |
|---|---|
| Ingredient | Composition |
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablet is manufactured e.g. as described in Formulation Example 1.

FORMULATION EXAMPLE 4
Capsules:

| | Composition Per Unit (mg) |
|---|---|
| Components | |
| Valsartan [= active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total tablet mass | 209.50 |

The tablet is manufactured e.g. as follows:

### Granulation/Drying

Valsartan and microcrystallin cellulose are spray-granulated in a fluidised bed granulator with a granulating solution consisting of povidone and sodium lauryl sulphate dissolved in purified water. The granulate obtained is dried in a fluidised bed dryer.

### Milling/Blending

The dried granulate is milled together with crospovidone and magnesium stearate. The mass is then blended in a conical srew type mixer for approximately 10 minutes.

### Encapsulation

The empty hard gelatin capsules are filled with the blended bulk granules under controlled temperature and humidity conditions. The filed capsules are dedusted, visually inspected, weightchecked and Guarantied until by Quality assurance department.

FORMULATION EXAMPLE 5
Capsules:

| | Composition Per Unit (mg) |
|---|---|
| Components | |
| Valsartan [= active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total tablet mass | 342.00 |

The formulation is manufactured e.g. as described in Formulation Example 4.

FORMULATION EXAMPLE 6
Hard Galatine Capsule:

| Components | Composition Per Unit (mg) |
|---|---|
| Valsartan [= active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| Total tablet mass | 130.00 |

### Formulation Example 7:

A hard gelatin capsule, comprising as active ingredient e.g. (S)-N-(1-carboxy-2-methylprop-1-yl)-N-pentanoyl-N-[2′(1H-tetrazol-5-yl)biphenyl-4-yl-methyl]amine, can be formulated, for example, as follows:

FORMULATION EXAMPLE 7
A hard gelatin capsule, comprising as active ingredient e.g. (S)-N-(1-
carboxy-2-methylprop-1-yl)-N-pentanoyl-N-[2′(1H-tetrazol-5-yl)bi-
phenyl-4-yl-methyl]amine, can be formulated, for example, as follows:

| Composition: | |
|---|---|
| (1) valsartan | 80.0 mg |
| (2) microcrystalline cellulose | 110.0 mg |
| (3) polyvidone K30 | 45.2 mg |
| (4) sodium lauryl sulfate | 1.2 mg |
| (5) crospovidone | 26.0 mg |
| (6) magnesium stearate | 2.6 mg |

Components (1) and (2) are granulated with a solution of components (3) and (4) in water. The components (5) and (6) are added to the dry granulate and the mixture is filled into size 1 hard gelatin capsules.

Components (1) and (2) are granulated with a solution of components (3) and (4) in water. The components (5) and (6) are added to the dry granulate and the mixture is filled into size 1 hard gelatin capsules.

FORMULATION EXAMPLE 8

| Component | Amount per Unit (mg) |
|---|---|
| Core | |
| fadrozole (hydrochloride), Hemihydrate | 1.035 ¹⁾ |
| Aerosil 200 (Silica aerogel) | 0.200 |
| Avicel PH 102 (Cellulose) | 37.300 |
| Cellulose-HP-M 603 (Hydroxypropyl | 2.000 |
| methylcellulose) | |
| Lactose ground | 53.965 |
| Magnesium stearate | 0.500 |
| Polyvinyl-polypyrrolidone XL | 5.000 |
| Weight of Core | 100.000 |
| Coating | |
| Cellulose-HP-M 603 (Hydroxypropyl | 1.837 |
| methylcellulose) | |
| Iron oxide, red, 172661 | 0.017 mg |
| ron oxide, yellow, 17268 | 0.017 mg |
| Polyethylene glycol 8000, in flakes | 0.333 mg |
| Talc, PH | 1.330 mg |
| Titanium dioxide | 0.466 |
| Weight of Coating | 4.000 |

¹⁾1.035 mg CGS 16 949 A hemihydrate is equivalent to 1.000 mg anhydrate.

What is claimed is

1. Use of a pharmaceutical composition comprising
(i) an aldosterone synthase inhibitor or a pharmaceutically acceptable salt thereof alone or in combination with,
(ii) an AT₁-receptor antagonist or an AT₁ receptor antagonist combined with a diuretic or, in each case, a pharmaceutically acceptable salt thereof and
(iii) a pharmaceutically acceptable carrier; for the prevention of, delay of progression of, treatment of a disease or condition selected from the group consisting of
(a) hypertension, congestive heart failure, renal failure, especially chronic renal failure, restenosis after percutaneous transluminal angioplasty, and restenosis after coronary artery bypass surgery;
(b) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and
(c) endothelial dysfunction with or without hypertension.

2. Use according to claim 1 wherein said AT₁-receptor antagonist is selected from the group consisting of valsartan, losartan, candesartan, eprosartan, irbesartan, saprisartan, tasosartan, telmisartan, the compound with the designation E-1 477 of the following formula 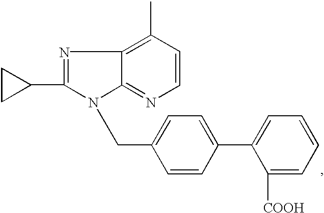
the compound with the designation SC-52458 of the following formula 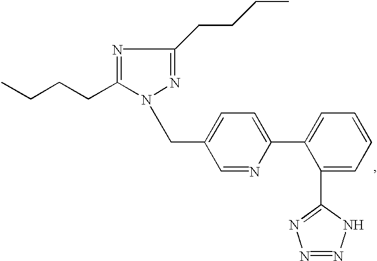
and the compound with the designation the compound ZD-8731 of the following formula 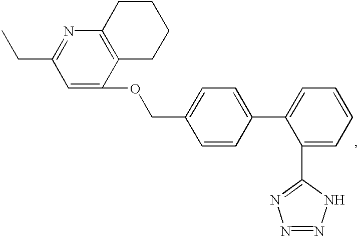
or, in each case, a pharmaceutically acceptable salt thereof.

3. Use according to claim 2 wherein said AT₁-receptor antagonist is valsartan or a pharmaceutically acceptable salt thereof.

4. Use according to any one of claims claims 1 to 3 wherein said aldosterone synthase inhibitor is selected from the group consisting of anastrozole, fadrozole (including the (+)-enantiomer thereof, and exemestane, or, in each case where applicable, a pharmaceutically acceptable salt thereof.

5. Use according to any one of claims 1 to 4 wherein said aldosterone synthase inhibitor is (+)-enantiomer of the hydrochloride of fadrozole of formula

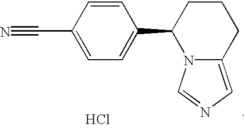

6. Use according to any one of claims 1 to 5 wherein the diuretic is hydrochlorothiazide.

7. Use of a pharmaceutical composition comprising an aldosterone synthase inhibitor or a pharmaceutically acceptable salt thereof, for the manufacture of a medicament for the prevention of, delay of progression of, treatment of a disease or condition selected from the group consisting of
(α) hypertension, congestive heart failure, renal failure, especially chronic renal failure, restenosis after percutaneous transluminal angioplasty, and restenosis after coronary artery bypass surgery;
(β) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and
(χ) endothelial dysfunction with or without hypertension.

8. A pharmaceutical composition comprising:
(i) an aldosterone synthase inhibitor or a pharmaceutically acceptable salt thereof either alone or in combination with,
(ii) an AT,-receptor antagonist or an AT₁ receptor antagonist combined with a diuretic or, in each case, a pharmaceutically acceptable salt thereof; and
(iii) a pharmaceutically acceptable carrier; for the prevention of, delay of progression of, treatment of a disease or condition selected from the group consisting of
(a) hypertension, congestive heart failure, renal failure, especially chronic renal failure, restenosis after percutaneous transluminal angioplasty, and restenosis after coronary artery bypass surgery;
(b) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis and remodeling following hypertension (antiproliferative effect of the combination), all these diseases or conditions associated with or without hypertension; and
(c) endothelial dysfunction with or without hypertension.

9. A method for the prevention of, delay of progression of, treatment of a disease or condition selected from the group consisting of
(a) hypertension, congestive heart failure, renal failure, especially chronic renal failure, restenosis after percutaneous transluminal angioplasty, and restenosis after coronary artery bypass surgery;
(b) atherosclerosis, insulin resistance and syndrome X, diabetes mellitus type 2, obesity, nephropathy, renal failure, e.g. chronic renal failure, hypothyroidism, survival post myocardial infarction (Ml), coronary heart diseases, hypertension in the elderly, familial dyslipidemic hypertension, increase of formation of collagen, fibrosis, and remodeling following hypertension (antiproliferative effect of the combination), all these diseases or conditions associated with or without hypertension; and
(c) endothelial dysfunction with or without hypertension; comprising administering to a warm-blooded animal, including man, a therapeutically effective amount of an aldosterone synthase inhibitor in free or pharmaceutically acceptable salt form.

10. Method according to claim 9 further comprising administering a therapeutically effective amount of an AT₁-receptor antagonist or an AT₁ receptor antagonist combined with a diuretic, in each case, in free or pharmaceutically acceptable salt form.
